(19) United States
(12) Patent Application Publication
(10) Pub. No.: US 2016/0178607 A1
(43) Pub. Date: Jun. 23, 2016

(54) Acquiring Reliable Data

(71) Applicant: CAMBRIDGE TEMPERATURE CONCEPTS LTD, Cambridge (GB)

(72) Inventors: Shamus Louis Godfrey HUSHEER, London (GB); Oriane Elisabeth CHAUSIAUX, Cambridge (GB)

(21) Appl. No.: 14/905,757

(22) PCT Filed: Jul. 21, 2014
(86) PCT No.: PCT/GB2014/052226

§ 371 Date: Jan. 15, 2016

(30) Foreign Application Priority Data
Jul. 19, 2013 (GB) 1312998.6

(51) Int. Cl. G01N 33/487; G01N 21/78

(52) U.S. Cl. CPC G01N 33/48792; G01N 33/48771; G01N 21/78

(57) ABSTRACT

A device (100) for determining a test result comprising: one or more sensors (104, 105) arranged to detect an assay result indicated by a test strip (101), such as a lateral flow test strip or a photometric test strip; a processor (102) configured to, in use, process signals from the one or more sensors (104, 105) so as to form an intermediate data set characterising the assay result and to process at least some of said intermediate data set to determine a test result; and an output unit (103); wherein the processor (102) is configured to cause the output unit to provide a human-readable representation (201) of the test result and assay data encoded in machine-readable form comprising at least some of the intermediate data set. Test strips can have an identification code (404) and the assay result can be optically read using a mobile device (301) such as a smartphone or a tablet. Assay result can as well be transferred to a remote data server (304).

### BACKGROUND OF THE INVENTION

This invention relates to devices for determining an assay result, systems for determining the level of an analyte indicated by an assay result, and systems for acquiring reliable assay data.

Lateral flow test strips have been available for many years for testing bodily fluids (e.g. blood, sweat, saliva and urine) for the presence of various analytes, such as hormones, drugs, blood glucose level etc. These tests can be useful for monitoring or detecting conditions such as pregnancy, fertility and diabetes. Such test strips are particularly convenient for testing for the presence of the hormone hCG in urine, which indicates whether a female is pregnant. More recently, digital testers have become available that increase the accuracy of the tests by providing a controlled environment in which the test strips are read by one or more optical sensors. These have become very popular as an accurate home pregnancy test.

Typically, digital testers comprise a slot for receiving a lateral flow test strip, within which LEDs are arranged to illuminate the active areas of the test strip along with optical sensors for detecting the absorption or reflection of the LED light. This allows characteristics such as the presence, size and intensity of the assay results indicated on the strips to be determined by the tester and presented to the user as a test result.

Much effort has been spent in this area improving the reliability of the binary results offered by digital testers. However, little thought has been given to capturing the data generated by an assay test and improving the quality and accuracy of a test result provided to a user.

### SUMMARY OF THE INVENTION

According to a first aspect of the present invention there is provided a device for determining a test result comprising:
one or more sensors arranged to detect an assay result indicated by a test strip;
a processor configured to, in use, process signals from the one or more sensors so as to form an intermediate data set characterising the assay result and to process at least some of said intermediate data set to determine a test result; and
an output unit;
   wherein the processor is configured to cause the output unit to provide a human-readable representation of the test result and assay data encoded in machine-readable form comprising at least some of the intermediate data set.

The intermediate data set could characterise the strength of one or more markings indicating the assay result. The strength of the one or more markings could indicate the assay result is identified by one or more of the intensity, size, shape or colour of the markings. The sensors of the device may be configured to identify the intensity, size, shape or colour of the markings by sensing one or more of the absorption, reflectivity, and luminance of the markings.

The intermediate data set could include one or more of:
raw or processed signals from the one or more sensors; and
representations of the strength of the assay result determined by the device.

The processor may be configured to cause the output unit to provide the assay data in a repeating loop.

The processor may be further configured to cause the output unit to provide encoded in machine-readable form calibration data stored at the device.

The device may further comprise one or more sensors for reading an identifier of a test strip and the processor is configured to cause the output unit to provide at its output unit encoded in machine-readable form an identifier of a test strip located in the device.

The intermediate data set could characterise the assay result at two or more points in time so as to characterise the development of the assay result.

The test strip may be integral with the device or the device may be configured to receive the test strip such that its sensors are directed to the assay result.

The output unit could include a display screen and the assay data is provided on the display screen as a sequence of machine-readable codes.

The output unit could include an optical or acoustic emitter for signalling the assay data in the machine-readable form.

According to a second aspect of the present invention there is provided a system for determining the level of an analyte indicated by an assay result comprising:
a test strip for providing an indication of an assay result;
a test device adapted for receiving the test strip, the test device being configured to, in use, detect an assay result provided at the test strip by forming an intermediate data set characterising the strength of the assay result and processing at least some of said intermediate data set to determine a test result for provision to a user; and
a data processing device arranged to receive from the test device assay data representing at least some of the intermediate data set and calibration data for the test device;
   wherein the data processing device is configured to process the assay data in dependence on the calibration data so as to infer a level of analyte indicated by the assay result independently of the test result determined by the test device.

The data processing device may be configured to infer the level of analyte indicated by the assay result at the test strip by using the calibration data to convert the strength of the assay result indicated by the assay data into a corresponding analyte level.

The calibration data could express the relationship between the strength of an assay result and the analyte level it represents for the combination of the test device and test strip.

The test device could include one or more sensors arranged for detecting the assay result and the intermediate data set comprising one or more of:
raw or processed data from the one or more sensors;
sensor data calibrated using calibration data held at the test device; and
data representing measures of one or more of the strength, colour, intensity, absorption, and reflectivity of the assay result as detected by the test device.

The data processing device may be further configured to receive from the test device an identifier of the test device and to use the identifier to validate the test device at a database accessible to the data processing device, the data processing device being configured to discard or otherwise not make use of the assay data if the test device is not successfully validated at the database.

The test device may be not successfully validated if the database indicates that the test device is out of date, has been recalled by the manufacturer, or has been used more than a predetermined number of times or for more than a predetermined period of time.

The data processing device may be a computer server arranged to receive the assay data and calibration data by means of an intermediate device arranged to visually capture the assay data and calibration data from the test device by means of a digital camera and forward that data to the data processing device.

The intermediate device may be a portable device configured to capture the assay data and calibration data from the test device by means of its camera, and the test device is configured to provide the assay data and calibration data at its display screen as a sequence of machine-readable codes.

The data processing device may be remote to the test device.

According to a third aspect of the present invention there is provided a system for determining the level of an analyte indicated by an assay result comprising:
a test strip for providing an indication of an assay result for an analyte;
a test device having an identifier and being adapted for receiving the test strip, the test device being configured to, in use, detect an assay result provided by the test strip by forming an intermediate data set characterising the strength of the indication provided by the test strip and processing at least some of said intermediate data set to determine a test result; and
a data processing device arranged to receive from the test device assay data that includes representations of at least some of the intermediate data set and identifier data that includes a representation of the identifier of the test device; wherein the data processing device is configured to use the identifier data to lookup calibration data for the test device in a database accessible to the data processing device and to process the assay data in dependence on those calibration data so as to infer the level of analyte indicated by the assay result independently of the test result determined by the test device.

The data processing device may be configured to infer the level of analyte indicated by the assay result at the test strip by using the calibration data to convert the strength of the assay result indicated by the assay data into a corresponding analyte level.

The calibration data could express the relationship between the strength of an assay result and the analyte level it represents for the combination of the test device and test strip.

The test device may be configured to use the calibration data in its processing of the intermediate data set to determine a test result.

The intermediate data set could comprise values representing the output of one or more sensors of the test device.

The test strip could bear an identifier and the test device further comprises a sensor for reading the test strip identifier, the test device being configured to provide the test strip identifier with the assay data, and the data processing device being configured to use the test strip identifier in the database lookup so as to identify calibration data for the test strip.

The test device may be configured to provide the assay data to the data processing device as a sequence of machine-readable codes communicated by one or more of optical, acoustic or electromagnetic means.

The system could further comprise an intermediate device arranged to receive the sequence of machine-readable codes from the test device and relay the assay data to the data processing device over a network.

According to a fourth aspect of the present invention there is provided a system for acquiring reliable assay data comprising:
a test strip for providing an indication of an assay result for a predetermined analyte, the test strip bearing an identification code of the strip and a set of calibration markings;
intermediate apparatus operable to capture the identification code and an assay image of an assay result from the test strip; and
a data processing device arranged to receive the identification code and the assay image from the intermediate device and use the identification code to authenticate the test strip in a database accessible to the data processing device;
   wherein the data processing device is operable to process the assay image to identify the level of analyte from the strength of the indication of the assay result relative to the calibration markings.

The assay image could include the identification code and the data processing device is configured to process the assay image to extract the identification code.

The intermediate apparatus is may be configured to capture the identification code and assay image by means of a digital camera.

The intermediate apparatus may be configured to provide an identifier of its type to the data processing device and the data processing device having access to a second database comprising calibration data for a plurality of types of intermediate device, the data processing device being configured to use the identifier to lookup calibration data for the digital camera of the intermediate apparatus and to process the assay image in dependence on that calibration data.

The calibration markings could define a range of strengths of the assay result so as to allow the data processing device to determine the strength of the indication of the assay result in the assay image by comparison of the assay result to the calibration markings.

The data processing device may be configured to make use of the level of analyte only if the test strip is successfully authenticated.

### DESCRIPTION OF THE DRAWINGS

The present invention will now be described by way of example with reference to the accompanying drawings, in which:

FIG. 1 a is a schematic diagram of a test device in accordance with the present invention.

FIG. 1b is a schematic diagram of a disposable test strip in accordance with the present invention.

FIGS. 2a and 2b are illustrations of a display screen providing a human-readable assay result and a machine-readable code.

FIG. 3 is a schematic diagram of a system configured in accordance with the present invention.

FIG. 4 is an illustration of a lateral flow test strip suitable for use with apparatus of the present invention.

### DETAILED DESCRIPTION OF THE DRAWINGS

The following description is presented to enable any person skilled in the art to make and use the invention, and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

The present invention relates to devices and systems for determining assay results and levels of analyte indicated by test strips, such as lateral flow test strips. Test strips can be any kind of carrier (e.g. paper) of any shape supporting an assay test that generates an assay result detectable from the strip. Test strips are widely used to detect the presence of drugs, hormones or other natural or artificial compounds in bodily fluids, and form the basis of most home pregnancy test kits. The term test device is used herein to refer to electronic devices for determining assay results from test strips. Test devices may or may not themselves be disposable in the sense that they are intended to be thrown away after a certain period of time, a certain number of uses, or when the battery dies. The term data processing system is user herein to refer to any kind of computer system, including servers, personal computers such as laptops, and portable devices such as smartphones and tablets. Assay results can be a result of a test for a specific analyte or a result generated from a combination of assay tests for different analytes. Depending on the analyte and assay performed, an assay result can be indicated by the active markings of a test strip by the presence or lack of a marking, the colour of a marking, its luminensence, the intensity of a marking being above or below a predetermined threshold, the shape of a marking, or any other indication as appropriate to the assay being performed and the analyte involved.

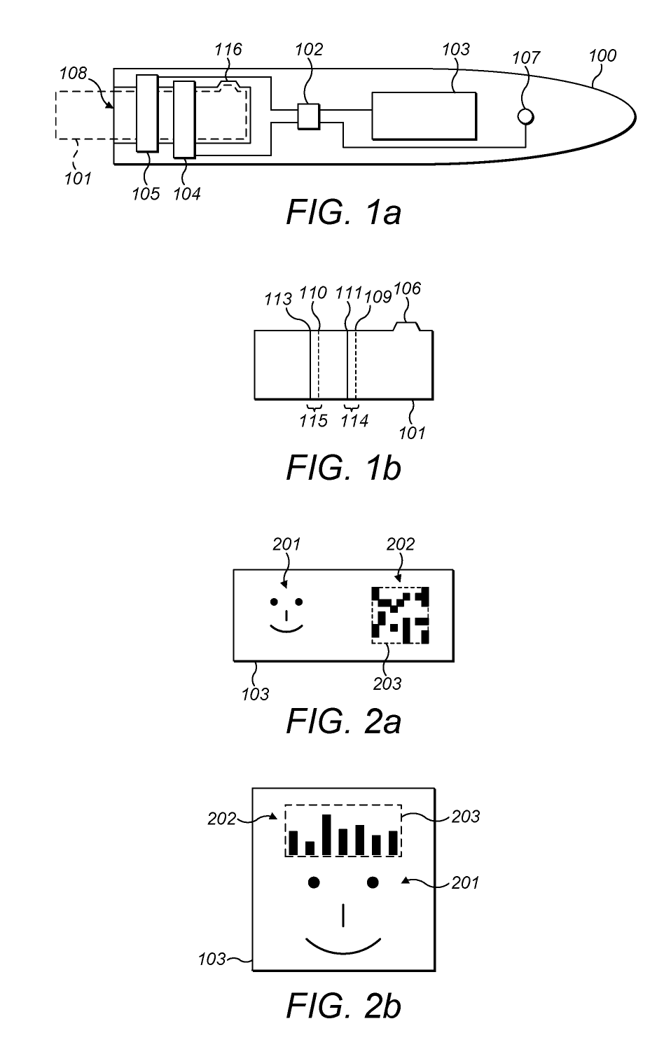

A schematic diagram of a test device is shown in FIG. 1a. Test device 100 comprises a slot 108 for receiving a test strip 101 and sensors 104 and 105 arranged so as to detect assay results indicated within predetermined zones of a strip present in the device. Any suitable means for receiving a test strip could be used other than a slot—for example, the test device could have a flap in its outer shell which is lifted so as to allow a test strip to be placed into the device. In other examples, the test strip and test device are provided as a single unit, with the test strip being integrated into the test device. An exemplary test strip 101 is shown in FIG. 1b that comprises active zones 114 and 115 in which lines or other markings appear when the corresponding analyte is detected. Typically, test strip 101 would be a lateral flow test strip intended for the detection one or more predetermined analytes in the bodily fluids of a user. For example, test strip 101 could be pregnancy test strip configured to develop one or more lines of a predetermined colour when the presence of one or more hormones associated with the onset of pregnancy are detected, such as human chorionic gonadotropin (hCG). In another example, the test strip could be for detecting blood glucose so as to aid in the management or detection of diabetes.

In the example shown in FIG. 1, there are two optical sensors 104 and 105, but there could be any number of sensors or groups of sensors of any type, as appropriate for the test strips with which the device is to be used. Each sensor or group of sensors could be arranged to detect a different assay result. For example, sensor 104 is arranged to detect the assay result indicated by the presence of line 109 in zone 114 of test strip 101 and sensor 105 is arranged to detect the assay result indicated by the presence of line 110 in zone 115 of test strip 101. Typically, assay results 109 and 110 would indicate the presence of different analytes, such as different hormones on a pregnancy test strip. Some of the assay results could be control lines configured to indicate whether the test being performed is valid.

The test device would typically be configured to detect the assay results and present a test result to the user a predetermined length of time after a test strip is inserted into the test device, or following a button being pressed by the user. This is because the assay results of lateral flow tests generally develop over a known period of time (e.g. 5 minutes). The test device could be configured to vary the length of time it waits for each assay result to develop, for example in response to environmental conditions such as temperature, or in response to inputs from the user at the test device.

Test device 100 further includes a processor 102 for receiving signals from the sensors and processing those signals so as to provide to a user by means of output device 103 an assay result determined from one or more assays performed on a test strip located in the device. In FIG. 1, the output device is a display screen and the assay result could be in the form of a verbal message, a symbol (such a smiling face 201 in FIG. 2a, which could be suitable for a pregnancy test device), or any other suitable visual indication. Output unit 103 could alternatively or additionally provide an audible, tactile or vibrational output to a user.

To give an example, in the case that test device 100 is a digital pregnancy tester, the device would be configured for use with lateral flow strips 101 arranged to detect the presence of one or more analytes in a bodily fluid of a user (e.g. urine) that are indicative of pregnancy. Such lateral flow strips typically indicate pregnancy through the presence of one or more coloured lines in the active zones 114, 115 of the strip. Usually one of the lines is a control line. The optical sensors 104, 105 of the pregnancy tester are arranged such that, when a lateral flow strip is located in its receiving slot, the optical sensors lie over the active zones of the strip and detect whether the respective coloured lines are present. If the lines are present, the pregnancy tester indicates to the user that they are pregnant.

Other assay tests work in other ways. For example, lateral flow strips for detecting ovulation typically rely on the intensity of a coloured line indicative of the level of luteinizing hormone exceeding a threshold intensity. In order to determine the assay result, a test device configured for use with lateral flow strips for detecting ovulation would determine the intensity of the coloured line from the output of its optical sensors and provide a positive assay result to the user if that intensity is above a predetermined threshold.

The sensors of the device could work in any suitable manner so as to detect the assay results indicated by a test strip. Typically the sensors would be optical sensors. For example, optical sensors can be arranged to detect the presence or intensity of an assay result on a test strip through measurements of reflectivity of light from one or more sources (looking for either high absorption or high reflectivity in the active zone as appropriate), measurements of absorption (e.g. high or low transmittance of light) from one or more sources. Suitable sources include LEDs and it can be advantageous to use certain frequencies of light, e.g. those that interact most strongly with the assay result markings. These frequencies could lie in any part of the electromagnetic spectrum, including visible light, ultra-violet and infra-red. Other sensors types could equally be used to detect assay results, as appropriate to the test strips used—for example, magnetic sensors for detecting an assay result indicated by a band of magnetic particles.

Test strip 101 would typically include one or more physical indents or notches 106 designed for engagement with corresponding parts 116 of receive slot 108 of the test device. Such physical indents or notches can be used to ensure that only test strips intended for use with the test device can be inserted into the test device, and/or to indicate to the test device that a test strip has been inserted (e.g. the logic of the test device could power up in response) and/or to indicate to the test device the type of strip that has been inserted (e.g. to cause different sensors of the test device to be activated and/or to cause processor 102 to process the signals from the sensors in the appropriate manner).

Test strip 101 could further comprise one or more calibration markings 111, 113, suitably with a calibration marking being provided for an assay result in the active zone of that test result (e.g. calibration marking 111 is provided for assay result 109). Calibration markings can be used by the device as a reference such that the processor assesses the presence or intensity of an assay result relative to its calibration marking, rather than from the absolute values of each sensor for the assay results alone. This helps to minimise the effect of changes in sensor output due to, for example, changes in temperature and variations in LED output and sensor calibration over time. The use of calibration markings on test strips can mean the precision and level of calibration of the components in the device can be lower and hence reduce its cost. In some embodiments, the test device is disposable.

Test device 101 is provided with an output device to allow the provision of machine-readable data from the test device to a data processing endpoint, such as a remote server, for storage or processing. The output device is preferably a display screen 103 of the device or other optical output device, such as an infra-red LED 107. Alternatively, the output could be acoustic, including ultrasound (e.g. the output device could be an ultrasonic sounder whose output is modulated so as to encode assay data in a machine-readable form). In less preferred embodiments it could be a wireless radio connection but the logic required to support such connections is generally not compatible with device 100 being a low-cost, often disposable device.

An example of a display for conveying human-readable and machine-readable data is shown in FIG. 2a. Display screen (e.g. an LCD) 103 is configured to output a human-readable test result 201, which in the example shown is a smiling face (this can be suitable if the test device is a pregnancy tester). This is a result which is typically available on prior art devices, such as digital pregnancy testers from ClearBlue and First Response. Any suitable test result 201 could be provided as appropriate to the assays being performed on the test strip. The test result could be, for example, a symbol, a message (e.g. indicating a positive or negative result), or a value (such as a level of blood glucose).

The display screen of device 100 further provides a machine-readable output 202 which is in a form that cannot be read directly by the user and which carries information relating to one or more characteristics of the assay results indicated on the test strip and from which the levels of one or more analytes can be inferred. In this embodiment the machine-readable output of the test device is optical; in alternative embodiments the output could be in any other form, including acoustic/ultrasound or electromagnetic in nature. The characteristics of the assay results could be raw or processed values from the sensors of the device, or data derived therefrom by the processor. The characteristics could be, for example, intensity, size, shape, or colour of an assay result as determined by the sensors of the device. Any other characteristic indicating the strength of an assay result could be used.

A second example is shown in FIG. 2b, in which the display 103 combines the human and machine readable elements into a common output. In this example, one or more parts of the image conveying the test result (smiling face 201) are modulated so as to provide the machine-readable codes. In this case, pixels of the display screen are modulated above the face in a manner which appears to the user as a representation of hair but actually encodes assay data so as to allow reading of that data by a suitable machine (e.g. by means of a smartphone equipped with a camera). This approach helps to keep the interface clean and avoids confusing the user.

In order to generate the human-readable test result 201, the test device processes signals from the sensors 104 and 105 to determine the assay results indicated by test strip. As has been discussed, assay results on test strips indicate the level of an analyte through one or more of the intensity, colour, size or shape of markings that develop on the strip in the presence of the analyte. The test device is configured to capture such characteristics of the assay results on a test strip so as to allow the processor to determine a test result from the strength of the assay results. For example, a test device might return a positive test result if the strength of an assay result indicates that the levels of the analytes are above a predetermined threshold and therefore represents e.g. a positive urine test for a drug, that LH exceeds a predetermined threshold and the test female is ovulating, or that blood glucose levels are above a predetermined threshold. Alternatively, the test device could provide a value as the test result, for example a value derived by the processor from the characteristics of an assay result on the basis of a known relationship between the strength of the assay result and the level of the respective analyte.

Typically, device 100 would hold calibration values so as to allow the processor 102 to accurately relate the output of the sensors to the characteristics of the assay result indicated on a test strip inserted into the device. Calibration values would typically characterise the relationship between the physical output of the sensors and the variable to be measured. For example, in the case of a pregnancy test strip and device, a calibration value might connect the relative voltage output of a pair of photodiodes—a first photodiode directed to a test area of the strip and a second photodiode directed to a calibration area of the strip—with a measure of the concentration of a hormone indicative of the onset of pregnancy. Significantly more complex calibration arrangements are possible, depending on the device. For example, a device with a temperature sensor could potentially use the output the temperature sensor and suitable calibration values to perform a temperature-dependent correction on the assay test result as detected by optical sensors. Calibration values for the device would typically be set during manufacture and used at runtime by the device in the formation of the test result.

Processor 102 is configured to cause the output unit of the device (in this case display screen 103) to signal at least some of the characteristics of an assay result to a data processing device. This is achieved through the use of machine-readable codes carrying information representing characteristics of assay results indicated on test strip 101. In the preferred embodiment, machine-readable output 202 is provided as a sequence of two-dimensional codes at display screen 103—e.g. a series of codes represented by patterns of pixels that are displayed in sequence according to a predetermined timing pattern. Alternatively, individual pixels or groups of pixels within the machine-readable output area 203 could be switched on and off, or have their intensity, colour or position modulated according to predetermined and possibly different timing patterns—for example, it could be an icon provided to the user to indicate the test result at the device which is itself modulated in order to communicate the assay result in a machine-readable manner.

The machine-readable codes 202 are provided within a predefined area 203 of the display screen so as to allow the codes to be captured by a digital camera as a series of images or as a video and processed so as to extract the data encoded therein. The predefined area 203 could be one and the same as the human readable output 201—e.g. the machine readable output could be encoded as a modulation of an icon used to indicate the result to the user. The camera could be provided at the data processing endpoint, or at any intermediate device such as a webcam, digital camera, tablet or smartphone. The test device is preferably configured to display both the test result 201 and encoded data 202 simultaneously as soon as the device has generated the test result. Alternatively or additionally, the test device can be configured to provide the machine codes in response to an input from the user (e.g. a button press) and in this case the test device could provide data stored at the device that relates to one or more test strips that have been read by the test device.

Depending on the assays involved, it can be advantageous to configure the test device to provide encoded data representing the characteristics of assay results as they develop on the test strips over time. Such information could be provided at the machine-readable output once the test result has been formed or even before the simple test result is available to the user. The rate at which the assay results develop and their characteristics over time can provide useful information relating to the analyte itself. This information can be processed at a data processing system arranged to receive the data from the test device so as to improve the accuracy, sensitivity or information content of a result generated at the data processing system.

It is advantageous if the machine-readable codes are repeated in a loop for some period of time or in response to some event (such as the user pushing a button on the test device) so as to allow a user to capture the entire information content of the codes without requiring the intermediate device to trigger the output of the machine-readable code (necessitating that bidirectional communication is supported between the devices) or that the user begin capturing data from the test device at precisely the correct moment in time. Other techniques could be used for improving the reliability of communications from the test device to the data processing device. For example, the transmission of the most important data could be prioritised in transmissions by the test device—e.g. such data could be transmitted more frequently or repeated more often in a looping sequence of machine-readable codes. Error correction could be used so as to allow the recovery of assay data in circumstances of partial data loss.

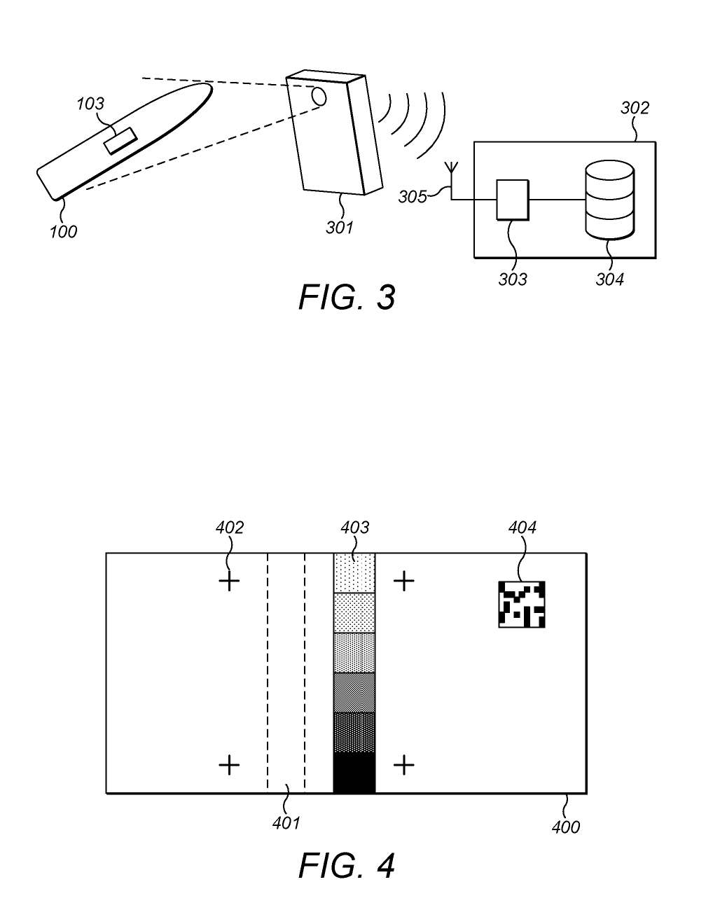

Advantageously and as shown in FIG. 3, the camera would be the camera of a portable device 301 (e.g. a smartphone, tablet or laptop) provided with an application configured to direct a user to capture the codes from a test device and upload the captured images or video to a data processing system configured to make use of the information contained therein. In less preferred embodiments the smartphone could be the consumer of the encoded data and extracts the data from the codes for storage at the smartphone/online, or for use in processing performed at the smartphone. However, in medical applications this can require the smartphone and/or its application to be medically approved and it is therefore preferred that any intermediate device used to capture the machine-readable codes merely relays the encoded data to the intended data processing endpoint 302 configured in accordance with any necessary medical approvals.

The data processing endpoint 302 includes a receiver 305 (e.g. a wired or wireless connection to a network) by means of which it receives the encoded data from the portable device, a processor 303 for decoding the received data, and a data store 304 for storing the received data and/or the results of processing performed on that data.

Test device 100 is typically a low-cost, disposable device and the preferred embodiment provides a mechanism whereby information characterising the assay results of a test strip can be provided by the device for use at other data processing devices without significantly increasing the complexity and cost of the device. A typical test device already includes a processor for determining a test result from the output of its sensors and driving its LCD screen. Such low cost processors generally already provide sufficient logic to allow them to be programmed to encode data representative of the assay result characteristics determined by the sensors according to a simple protocol.

The output unit for the machine readable data could alternatively be separate to the output unit for the test result. For example, the encoded data could be provided at optional LED 107, with the processor being arranged to cause the output of the LED to be modulated in accordance with a simple protocol. Preferably the LED would be an infra-red LED because the sensors of digital cameras are typically sensitive to IR light and the modulation of the output of the LED would not be as distracting to the user as light in the visible portion of the spectrum. Driver logic for LEDs can be readily incorporated into the logic available in low cost, disposable test devices. In less preferred embodiments, the output unit for the encoded data outputs the data by, acoustic (including ultrasound), NFC or other wireless means.

The encoded data could include the test result determined by the test device.

The test device is preferably configured to provide its calibration values (e.g. those stored at the device during manufacture) in the encoded data 202 provided at its machine readable output. The test device can provide the calibration values along with the raw sensor outputs of the device. Most preferably, the test device provides the raw sensor outputs of the device at a plurality of points in time whilst the assay result is being formed at the test strip. This allows the data processing endpoint 302 to receive data describing the evolution of the assay result at the test strip and can allow the data processing endpoint to, through the use of more complex algorithms than run at the test device, more accurately infer the analyte level indicated by an assay result than can the test device, and/or to provide information indicating the confidence of the assay result reported by the data processing endpoint/test device.

The calibration values would typically specify the expected relationship between analyte level and sensor output for the test device. The calibration values could further be particular to a batch of test strips provided with the test device, or test strips of a particular type.

Generally, test devices are intended for use with specific types of assay strips that are configured to provide assay results in the form of a particular types of markings in predetermined positions. By arranging that the calibration values capture the characteristics of both the test device sensors and the test strips on which the test device operates, and by providing those calibration values to a data processing endpoint, the data processing endpoint can accurately infer analyte levels and other quantitative information based on the simple assay results of a cheap disposable test strip. A data processing endpoint could calculate analyte levels from the encoded data provided by the test device in a similar manner to the calculations that would typically be performed by the test device in order to form the test result. Preferably however, since the data processing endpoint would generally have significantly greater processing power at its disposal as well as potentially access to other data sets (perhaps describing more accurately the relationship between particular output sensor values and the variable under measurement), the data processing endpoint could perform more complex analyses of the assay result characteristics provided by the test device.

It is advantageous if the test device is allocated a unique identifier (e.g. during manufacture) which it provides in the encoded data at the machine readable output. This allows the data processing endpoint (which might be using analyte levels calculated from the encoded data in sensitive analyses to determine medical conditions or problems) to authenticate the test device in a database of test devices (e.g. 304). For example, the data processing endpoint could use the device identifier to look up whether the test device is approved for use in the test concerned, is out of date, has been recalled by the manufacturer, has been used more than a predetermined number of times or over too long a period (which may mean its calibration is no longer good enough), or is otherwise not valid for use. This mechanism allows the data processing endpoint to verify that the encoded assay data received by the data processing endpoint is reliable. Such an authentication database could be provided by the manufacturer of the test device.

Data processing endpoint 302 could be configured to perform any suitable processing on the encoded assay data received from a test device but is particularly useful for providing information to data processing endpoints configured to perform data processing relating to fertility or pregnancy. Test strips are available for various hormones relating to fertility and pregnancy, including LH, oestrogen, hCG, progesterone, AMH and FSH and, as is well known in the art, the level of these hormones can be very useful in determining information such as whether and when a female is fertile, whether and medical conditions relating to fertility are indicated, the point of ovulation, whether a female is pregnant and how long the female has been pregnant, as well as many other parameters. This hormone level information, as calculated at the endpoint from assay data provided to it according to the principles described herein, can be fed into algorithms running at a data processing endpoint in order to improve the accuracy of predictions and models running at the endpoint. For example, a server running a fertility monitoring system arranged to determine the point of ovulation from the time-varying basal body temperature data of a female can use assay data identifying hormone levels to improve the accuracy of predictions or fill in gaps in the temperature data.

It is particularly advantageous if a database accessible to the data processing endpoint stores calibration parameters for the test device. This could provide an alternative to the data processing endpoint receiving calibration parameters from the test device and/or the database could provide additional calibration parameters so as to allow the data processing endpoint to perform more complex processing of assay data received from the test device. Such calibration parameters could be particular to a test device/a batch of test devices (e.g. as derived from testing a device/a device of a batch at production time) and stored at an authentication database and indexed using an identifier of the test device.

These calibration parameters could be different/a superset of any calibration parameters stored at the test device. For example, the calibration parameters could express the relationship between the output of the test device sensors over time as the assay result evolves with a measure of the concentration of the analyte that the test strips are adapted to detect. Thus, by arranging that the test device provide data describing the evolution of sensor readings over time to the data processing endpoint (as described above) and providing the data processing endpoint with suitable calibration parameters, the data processing endpoint could generate significantly more information for the user. For instance, such an arrangement could allow the data processing endpoint to generate an estimate of the concentration of the analyte, not just an indication as to whether or not the analyte is present at a concentration greater than some predefined level. The level of luteinising hormone (LH) by an assay test would be useful, for example, at a data processing system configured to provide information relevant to a woman suffering from Polycystic Ovary Syndrome (PCOS).

Furthermore, because the evolution of an assay result can vary with environmental parameters, such as temperature and humidity, it can be advantageous to (a) provide the test device with appropriate sensors (e.g. a temperature sensor) and (b) provide calibration parameters that describe the dependence of the evolution of the assay result on that respective variable (in this case, temperature).

It can be advantageous if the data processing endpoint has access to calibration parameters which are specific to a given test strip, type of test strip or batch of test strips, e.g. those test strips sold with the test device as a kit. This allows, for instance, processing to be performed in dependence on the particular characteristics of the test strips provided with the device. More generally, test strips can be allocated an ID (which could be unique to the strip or a batch to which the strip belongs). This can allow a data processing endpoint to retrieve from a calibration store the calibration parameters particular to that strip, or the batch of strips to which that parameter belongs. The ID of a strip could for example be printed on the test strip so as to allow the user to enter the ID of the strip into an intermediate device (such as a smartphone) in any suitable manner, including by manual entry or taking a photo of an ID printed on the strip (which could be a barcode, QR code or suchlike). The ID could be provided on a strip in any manner as described below for identifier code 404 in FIG. 4.

The performance of test strips can vary from one batch or manufacturer to another, particularly with respect to their variation in performance over time. Providing a database of calibration values for test strips available for use with a test device can therefore improve the accuracy of a data processing endpoint which can access the database. Calibration data can be provided to the database through the testing in controlled conditions of one or more test strips from each batch; by performing that testing on a set of strips from a batch for some period of time (e.g. several months) following their production, the variation in performance of the test strips over time can be captured and stored in appropriate calibration values at the calibration database. A batch could be any set of test strips, including all test strips produced by a particular manufacturer or factory, and need not correspond to batch numbers used for product tracking or other commercial purposes.

On receiving the encoded assay data and device identifier from a test device, the data processing endpoint is configured to retrieve calibration parameters for the device from the database and perform processing of the assay data in dependence on those calibration parameters. This has several benefits in addition to the specific benefits discussed above for certain calibration parameters. Firstly, it ensures that only approved data processing endpoints granted access to the authorisation database can accurately process the assay data made available by the device. This is important to ensure that a user receives reliable information (which could be of a medical nature) from an approved chain of devices (from test strip to test device through to data processing endpoint). Secondly, it negates the need for the test device to transfer the calibration parameters each time it provides encoded assay data at output 202, reducing the amount of data that must be transferred and hence the time required. Thirdly, the calibration parameters held at the database can be modified over time in response to the expected effect of changes to the calibration of the device over time. And fourthly, the calibration parameters can be updated in response to improvements in the understanding of the relationship between assay result (at least as detected by the sensors) and analyte level.

The calibration parameters could be stored in the database by the device manufacturer on the device being calibrated (typically during manufacture).

Importantly the processing performed at the data processing endpoint is performed on data representing the characteristics of test strip assay results independently of the processing performed at the test device in order to form the test result presented by that device to the user. This ensures that the data processing endpoint is not constrained by the low processing power available at the test device.

The test device could be further configured to encrypt the assay data for transmission to a data processing endpoint by means of output 202. The test device encrypts the assay data using an encryption key stored at the device prior to or in the same step of encoding the assay data for transmission over output 202. Any suitable encryption algorithms could be used, including symmetric key and public key encryption systems. Preferably the encryption algorithm used is simple to implement at the limited logic available at the test device. Suitable ciphers include RC4 or a Tiny Encryption Algorithm.

The test device could be configured to provide its identifier in unencrypted form in the encoded data for transmission over output 202. This allows a data processing endpoint with access to authentication database 304 to use the device identifier as a lookup into the database in order to retrieve the decryption key for the test device from which the data has been received. In this manner, only those data processing endpoints granted access to the authentication database can acquire the assay data provided by a test device, hence verifying that both the test device and data processing endpoint form part of a reliable chain of authorised devices.

On performing a lookup for a device encryption key, the data processing endpoint could additionally perform device verifications as discussed above, and/or could retrieve calibration parameters for the device. The use of encryption at the test device means that test devices which have, for example, been recalled due to a fault, or which have served their useful life, can effectively be prevented from making their potentially erroneous data available to data processing devices by removing or otherwise blocking the corresponding decryption keys at the database.

In the case that an intermediate portable device (such as a smartphone, tablet or laptop) is used to capture the encoded assay data from the machine readable output of the test device, calibration parameters for the image acquisition means (e.g. digital camera) of that intermediate device would preferably be provided by that device to the data processing endpoint so as to allow the data processing endpoint to correct for the particular characteristics of the image acquisition means. Alternatively, the calibration parameters for the image acquisition means could be retrieved by the data processing endpoint from an online database (possibly the authentication database) using an identifier of the intermediate device. For example, a database could be provided with calibration parameters for the cameras of a range of different smartphones so as to allow for the correction of the particular characteristics of each of those smartphones. Calibration parameters for a digital camera could include parameters to correct for various types of optical aberration, including colour and/or spatial distortion.

In some examples, a data processing endpoint as discussed herein could be configured to acquire assay results directly from a test strip by means of intermediate apparatus comprising a digital camera and without requiring the use of a test device. Suitable intermediate apparatus could include a portable device of the type shown in FIG. 3, or a webcam or other imagine peripheral (such as a scanner) of the data processing endpoint. It is not important how the captured assay data is transferred from intermediate device to the data processing device and this may be achieved in any suitable manner, such as by a wireless or wired connection.

Suitable test strips generate a visual assay result and include calibration markings so as to allow a camera to both locate the expected position of an assay result and assess the strength of the assay result. An exemplary lateral flow test strip is shown in FIG. 4. Test strip 400 includes markings 402 which in this example are in the form of cross-hairs so as to allow the active zone of the test strip to be identified from an image of the test strip. The active zone of the test strip includes an assay result 401 which in this example is a coloured band that develops in the presence of the analyte to which the assay is directed.

The strength of the assay result indicated by a test strip could be determined from the image captured by the camera, perhaps relative to a calibration marking against which the strength of the assay result (e.g. in terms of its intensity or colour) can be compared. This approach can benefit from the use of calibration parameters determined for the camera, as described above.

It is particularly advantageous to provide a calibration band 403 defining the expected range of colours and/or intensities of assay result 401. This allows a camera to readily identify the strength of assay result 401 by comparing the assay result to the calibration band 403 so as to identify the closest match in the calibration band in terms of colour, intensity or other parameters. Using this mechanism allows the strength of the assay result and hence analyte level to be inferred at least to the resolution offered by the calibration band (i.e. if there are eight different levels in the calibration, at least eight different analyte levels could be inferred from the assay result). Preferably however, the intermediate apparatus is configured to interpolate between the calibration levels offered on the test strip so as to increase the resolution of the measurement of analyte level.

The test strip could provide one or more calibration bands, potentially relating to different parameters such as colour, brightness, saturation etc. Each band could comprise a number of blocks or other well-defined homogeneous zones, or a continuous gradient defining the expected range of the respective visual parameter.

Test strip 400 further comprises an identification code 404, which in the example shown is a two-dimensional barcode, but it could be any kind of visual code including typed characters. The data processing endpoint is preferably configured to receive an assay image comprising the assay result 401, calibration band 403, and identification code 404. Alternatively the identification code could be provided separately by the intermediate device, either as a separate image from which the code is to be extracted or as the extracted code if this is performed at the intermediate device. Processing of the assay image is however preferably performed at the data processing endpoint.

As is known the art, the assay image can be corrected so as to take account of variations in size, orientation and perspective of the assay result and calibration markings in the image due to the proximity and angle of the camera relative to the test strip when the image was captured.

On receiving the assay image, the data processing endpoint uses the identification code to authenticate the test strip in an authentication database (e.g. database 304 in FIG. 3). This allows the data processing endpoint to verify that the test strip is reliable by, for example, ensuring that the strip has not been used before, is within date and meets any necessary standards. If the test strip is successfully authenticated, the data processing device is configured to process the assay image in order to identify the strength of the assay result relative to the calibration band. Once the strength of the assay result is known relative to the range of expected variation indicated by the calibration markings, the data processing endpoint estimates the level of analyte indicated by that strength of assay result.

It is advantageous if the data processing endpoint is further configured to look up in the authentication database using the strip identifier the calibration parameters for the test strip that define the relationship for the strip between assay result strength and analyte level. This aspect therefore allows an accurate analyte level to be inferred from a simple image of an appropriately configured test strip.

It can be advantageous for the test device to include a sensor for reading an identifier of a test strip. The test device can then pass the test strip identifier to the data processing endpoint with the assay data so as to allow the data processing endpoint to lookup calibration parameters in an authentication database for the test strip itself, as well as any calibration parameters for the test device. Most preferably the identifiers of the test strip and device together identify a set of calibration parameters to be used to accurately infer analyte level from assay result strength.

In acquiring readings from its sensors, a test device configured in accordance with any of the teachings herein may form an intermediate data set characterising the assay result, the intermediate data set comprising one or more of: raw values from the sensors; sensor values which have undergone digital processing (e.g. filtering); sensor values which have calibrated against calibration values held at the test device; data representing the assay result (e.g. a measure of one or more of the strength, colour, intensity, absorption, and reflectivity of an assay result). The intermediate data set need not (and in any of the examples described herein does not) include the test result generated by the test device; it could comprise some or all of the data set from which the test device generates its test result.

Several examples and aspects of the present invention have been described herein. It is envisaged that any of the features of any of the examples or aspects of the present invention can be combined together and are not intended to relate solely to the examples or aspects of the present invention with respect to which those features have been described.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1.-12. (canceled)

13. A system for determining the level of an analyte indicated by an assay result comprising:
a test strip for providing an indication of an assay result;
a test device adapted for receiving the test strip, the test device being configured to, in use, detect an assay result provided at the test strip by forming an intermediate data set characterising the strength of the assay result and processing at least some of said intermediate data set to determine a test result for provision to a user; and
a data processing device arranged to receive from the test device assay data representing at least some of the intermediate data set and calibration data for the test device;
wherein the data processing device is configured to process the assay data in dependence on the calibration data so as to infer a level of analyte indicated by the assay result independently of the test result determined by the test device.

14. A system as claimed in claim 13, wherein the data processing device is configured to infer the level of analyte indicated by the assay result at the test strip by using the calibration data to convert the strength of the assay result indicated by the assay data into a corresponding analyte level.

15. A system as claimed in claim 13, wherein the calibration data expresses the relationship between the strength of an assay result and the analyte level it represents for the combination of the test device and test strip.

16. A system as in claim 13, the test device including one or more sensors arranged for detecting the assay result and the intermediate data set comprising one or more of:
raw or processed data from the one or more sensors;
sensor data calibrated using calibration data held at the test device; and
data representing measures of one or more of the strength, colour, intensity, absorption, and reflectivity of the assay result as detected by the test device.

17. A system as claimed in claim 13, wherein the data processing device is further configured to receive from the test device an identifier of the test device and to use the identifier to validate the test device at a database accessible to the data processing device, the data processing device being configured to discard or otherwise not make use of the assay data if the test device is not successfully validated at the database.

18. A system as claimed in claim 17, wherein the test device is not successfully validated if the database indicates that the test device is out of date, has been recalled by the manufacturer, or has been used more than a predetermined number of times or for more than a predetermined period of time.

19. A system as claimed in claim 13, wherein the data processing device is a computer server arranged to receive the assay data and calibration data by means of an intermediate device arranged to visually capture the assay data and calibration data from the test device by means of a digital camera and forward that data to the data processing device.

20. A system as claimed in claim 19, wherein the intermediate device is a portable device configured to capture the assay data and calibration data from the test device by means of its camera, and the test device is configured to provide the assay data and calibration data at its display screen as a sequence of machine-readable codes.

21. A system as claimed in claim 13, wherein the data processing device is remote to the test device.

22. A system for determining the level of an analyte indicated by an assay result comprising:
a test strip for providing an indication of an assay result for an analyte;
a test device having an identifier and being adapted for receiving the test strip, the test device being configured to, in use, detect an assay result provided by the test strip by forming an intermediate data set characterising the strength of the indication provided by the test strip and processing at least some of said intermediate data set to determine a test result; and
a data processing device arranged to receive from the test device assay data that includes representations of at least some of the intermediate data set and identifier data that includes a representation of the identifier of the test device;
wherein the data processing device is configured to use the identifier data to lookup calibration data for the test device in a database accessible to the data processing device and to process the assay data in dependence on those calibration data so as to infer the level of analyte indicated by the assay result independently of the test result determined by the test device.

23. A system as claimed in claim 22, wherein the data processing device is configured to infer the level of analyte indicated by the assay result at the test strip by using the calibration data to convert the strength of the assay result indicated by the assay data into a corresponding analyte level.

24. A system as claimed in claim 22, wherein the calibration data express the relationship between the strength of an assay result and the analyte level it represents for the combination of the test device and test strip.

25. A system as claimed in claim 22, wherein the test device is configured to use the calibration data in its processing of the intermediate data set to determine a test result.

26. A system as claimed in claim 22, wherein the intermediate data set comprises values representing the output of one or more sensors of the test device.

27. A system as claimed in claim 22, wherein the test strip bears an identifier and the test device further comprises a sensor for reading the test strip identifier, the test device being configured to provide the test strip identifier with the assay data, and the data processing device being configured to use the test strip identifier in the database lookup so as to identify calibration data for the test strip.

28. A system as claimed in claim 22, the test device being configured to provide the assay data to the data processing device as a sequence of machine-readable codes communicated by one or more of optical, acoustic or electromagnetic means.

29. A system as claimed in claim 22, the system further comprising an intermediate device arranged to receive the sequence of machine-readable codes from the test device and relay the assay data to the data processing device over a network.

30.-35. (canceled)
